(19) United States
(12) Patent Application Publication
(10) Pub. No.: US 2004/0161423 A1
(43) Pub. Date: Aug. 19, 2004

(54) Polymer modified anti-angiogenic serpins

(76) Inventor: Sanjeev Kumar (Mendiratta), Huntsville, AL (US)

Correspondence Address: WONG CABELLO LUTSCH RUTHERFORD & BRUCCULERI, LLP, 20333 SH 249, SUITE 600, HOUSTON, TX, 77070, US

(21) Appl. No.: 10/619,149

(22) Filed: Jul. 14, 2003

Related U.S. Application Data
(60) Provisional application No. 60/396,786, filed on Jul. 18, 2002.

(51) Int. Cl. C12N 9/99; A61K 38/43

(52) U.S. Cl. 424/94.1; 435/184; 525/54.1

(57) ABSTRACT

Methods and compositions for improving the angiogenesis-inhibitory effect of an anti-angiogenic serpin, or anti-angiogenic fragment thereof, by covalently linking a polymer moiety to the serpin such that the biological half-life of the serpin is extended for inhibition of diseases having a pathological angiogenic component including diabetic retinopathy, age-related macular degeneration, rheumatoid arthritis, endometriosis, psoriasis, juvenile hemangioma, and cancer.

### TECHNICAL FIELD OF THE INVENTION

The invention relates to agents for the inhibition of angiogenesis, and more particularly, to compositions and methods for improving the efficacy of anti-angiogenic proteins through covalent polymer modification. This application claims the benefit of priority to provisional U.S. application No. 60/396,786, filed Jul. 18, 2002.

### BACKGROUND OF THE INVENTION

Without limiting the scope of the invention, its background is described in connection with anti-angiogenic therapies. Angiogenesis, or the development of new blood vessels by sprouting from preexisting vessels, is initiated through the proliferation of endothelial cells. The rate of such proliferation in an adult is relatively low. However, certain diseases such as diabetic retinopathy, age-related macular degeneration, psoriasis, juvenile hemangioma, and rheumatoid arthritis are characterized by unregulated angiogenesis.

Angiogenesis is also a critical prerequisite for the sustained growth of tumors and their metastases. Therefore, inhibition of angiogenesis is considered an attractive strategy in the treatment of diseases having angiogenesis as a pathological component. Such a treatment strategy is anticipated to be broad spectrum in nature with applicability in the several diseases manifest by unregulated angiogenesis. In cancer, such an approach is anticipated to be applicable to more than one tumor type. Furthermore, most anti-angiogenic agents tested to date do not appear to have toxic side effects, making them even more attractive as drugs.

A number of anti-angiogenic proteins have been tested for efficacy in human clinical trials for cancer including endothelial cell inhibitors, matrix metalloproteinase inhibitors, VEGF antagonists, cytokines, and anti-integrin agents such as endostatin and angiostatin. Recently, anti-angiogenic properties have been identified in certain members of the serpin superfamily of proteins. Serpins were identified and so named for their capacity to act as serine protease inhibitors. However, the serpin superfamily also includes members that do not have protease inhibitory activity and the superfamily is defined by sequence and structural homology, rather than activity. Members of the serpin superfamily that have been found to exhibit anti-angiogenic activity include; pigment epithelium-derived factor (“PEDF”), maspin, antithrombin III, angiotensinogen and headpin.

PEDF was originally identified in the conditioned medium of cultured human retinal pigment epithelium cells as a neurotropic factor for cultured neuroblastoma cells. (Tombran-Tink et al., Invest. Opthalmol. Vis. Sci. 30 (8) 1770-7 (1989); U.S. Pat. No. 6,319,687). PEDF has recently been shown to be produced by differentiated ganglionic and Schwann cells within neuroblastomas, suggesting it may be responsible for spontaneous regression of the tumors. (Crawford et al 2001, J. Cell Sci. 114, 4421-4428). A primary physiologic role of PEDF appears to be suppression of ischemia induced neovascularization of the retina through the induced apoptosis of endothelial cells that form new blood vessels under the influence of vascular endothelial growth factor (“VEGF”). (Stellmach et al 2001, Proc. Natl. Acad. Sci. USA 98, 2593-2597).

Neovascularization is also a mechanism by which nascent cancer cells generate a vasculature sufficient to support tumor growth. As in the eye, neovascularization in solid tumors is thought to result from the action of angiogenic factors, such as vascular endothelial growth factor (“VEGF”), produced by the tumor cells. VEGF mRNA is up-regulated in the majority of human tumors and anti-VEGF antibodies have been shown to suppress the growth of a variety of tumor cell lines in nude mice. (Ferrara N. Breast Cancer Res Treat 1995;36(2): 127-37.). PEDF protein has been shown to be a potent inhibitor of the angiogenic effects of known angiogenic agents such as VEGF, FGF, PDGF, and IL-8 in in vitro assays. (Bouck et al. U.S. Pat. No. 6,288,024). Thus, Bouck et al. taught that PEDF can be provided as an anti-angiogenic agent either as a recombinant protein or as an expression cassette for endogenous production of PEDF.

Maspin is another non-inhibitory serpin normally present in mammary epithelial cells, but not in most mammary carcinoma cell lines and is known to have tumor suppressor activity. (Zou, Z., et al. (1994) Science 263, 526-529). In addition to mammary epithelia, maspin is expressed in placenta, prostate, and small intestine. Maspin expression is down-regulated in breast tumor cells as shown by both in vitro assays and by immunostaining of clinical specimens from breast cancer patients. Maspin expression has been reported to be consistently down-regulated during tumor progression. (Zhang and Zhang, Int J Oncol 2002 June;20(6):1145-50). In contrast, overexpression of maspin cDNA in human breast cancer cells inhibited the invasion of the cells in an in vitro invasion assay. Overexpression of maspin also significantly inhibited the primary tumor volume, auxiliary lymph node metastases, and lung metastases in nude mice when injected orthotopically into the tumor. (Xiao et al. Proc Natl Acad Sci USA 1999 Mar. 30;96(7):3700-5). Sager et al (U.S. Pat. No. 5,905,023) isolated DNA encoding maspin and observed that transfection of tumor cells with DNA encoding maspin inhibited growth of the tumor cells in vivo. Sager thus proposed the administration of maspin to patients having a carcinoma characterized by decreased expression of maspin and, alternatively, transfection of tumor cells as a gene therapeutic to inhibit tumor growth.

However, in the case of recombinant proteins, there exist formidable pharmacokinetic and economic disadvantages to chronic delivery. Many recombinant proteins are labile both in vitro and in vivo and have short biological half-lives. Short biological half-life can be a result of scavenging by the reticuloendothelial system, proteolytic degradation, clearance through the kidneys of molecules having a molecular weight less than 50,000 daltons (50 kDa), and in some cases, clearance or inactivation by binding of specific antibodies. Consequently, frequent administration is typically required in order to give sustained blood or tissue levels in the pharmacological range. For example, clinical trials with the anti-angiogenic agent endostatin involve continuous infusion of the recombinant protein.

What is needed are anti-angiogenic serpin compositions and methods that provide for increased bioactivity, such that these molecules can be effectively utilized in the treatment of conditions characterized by characterized by increased angiogenesis, such as diabetic retinopathy, age-related macular degeneration, psorisis, rheumatoid arthritis, endometriosis, and solid tumors.

### SUMMARY OF THE INVENTION

What is provided is a method of improving the angiogenesis-inhibitory effect of an anti-angiogenic serpin, or anti-angiogenic fragment thereof, by covalently linking a polymer moiety to the serpin such that the biological half-life of the serpin is extended. The method provides for inhibition of diseases having a pathological angiogenic component by administering in vivo an anti-angiogenic serpin, or fragment thereof, having a covalently linked polymer moiety. Diseases characterized by pathological angiogenesis include diabetic retinopathy, age-related macular degeneration, rheumatoid arthritis, endometriosis, psoriasis, juvenile hemangioma, and cancer.

In one embodiment, the anti-angiogenic serpin is selected from the group: PEDF, maspin, antithrombin III, angiotensinogen and headpin. In one embodiment, the anti-angiogenic serpin is PEDF. In another embodiment, the anti-angiogenic serpin is maspin.

In one embodiment, the polymer has a polyethylene moiety. In one preferred embodiment the polymer having a polyethylene moiety is selected from the group polyethylene glycol and poloxamer. The polymer may be covalently attached to the serpin using chemistries known to those of skill in the art. In one embodiment where polyethylene glycol is employed the polyethylene glycol moiety has a molecular weight of at least about 20 kDa. In one embodiment the polyethylene glycol is sulfonyl activated.

Compositions are provided having a modified anti-angiogenic serpin, or anti-angiogenic fragment thereof, wherein the serpin or fragment thereof is covalently linked to a polymer moiety. In one embodiment, the anti-angiogenic serpin is PEDF, maspin, antithrombin III, angiotensinogen or headpin. In one embodiment the polymer is selected from the group consisting of a polyethylene glycol and a poloxamer. In one embodiment the polymer is a monomethoxy polyethylene glycol that is sulfonyl activated.

### BRIEF DESCRIPTION OF THE FIGURES

FIG. 1 depicts the effects of local peritumoral delivery, 5 times weekly with unmodified PEDF at various concentrations, to tumors arising from subcutaneously implanted neuroblastoma cells.

FIG. 2 depicts the effect of intraperitoneal delivery of unmodified PEDF in tumor bearing mice.

FIG. 3 depicts the effect of intravenous delivery of unmodified PEDF in tumor bearing mice.

FIG. 4 depicts the effect of intravenous delivery of unmodified PEDF compared with polymer-modified PEDF in tumor bearing mice.

FIG. 5 depicts the fold increase in tumor size between day 6 to day 22 following systemic intravenous delivery of unmodified PEDF compared with polymer modified PEDF and controls.

FIG. 6 depicts the amino acid sequence of human PEDF.

FIG. 7 depicts the amino acid sequence of human maspin.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not limit the scope of the invention.

Anti-angiogenic agents are attractive candidates for the treatment of a number of disease conditions having pathology associated with angiogenesis for several reasons. There is little potential for cytotoxic or other physiologic side effects, in part because neovascularization in the adult is largely only associated with pathologic conditions. Tumor growth in excess of 1 to 2 mm is dependent on the formation of new vessels or neovascularization. An anti-angiogenic approach is considered to have the potential for broad spectrum activity against a variety of different tumor types. In addition, tumors are unlikely to develop drug resistance since they are not the direct targets of the anti-angiogenic regimen. In the case of diseases such as diabetic retinopathy, macular degeneration or endometriosis, where pathology is directly related to neovascularization, an anti-angiogenic approach directly targets a principal pathologic process. Recently, certain of the serpins have been shown to have powerful anti-angiogenic activity.

Serpins were originally identified and so termed as serine protease inhibitors. Protease inhibition results from the formation of irreversible 1:1 complexes between the serpin and the protease. Serpin superfamily proteins display a well-conserved tertiary structure that consists of 3 beta sheets and 8 or 9 alpha helices. (Huber, R. and Carrell, R. W. Biochemistry 28: 8951-8966, 1989). A critical portion of the molecule, the reactive center loop (“RCL”) or reactive site loop (“RSL”), connects beta sheets A and C and in some cases is the target for serine proteinase. More than 100 members of the serpin superfamily are known, including proteins having such disparate effects as C1esterase inhibitor (complement activation), antithrombin III (coagulation), protease inhibitor 7 (cell differentiation), pigment epithelium-derived factor “PEDF” (neurotrophic activity) and maspin (tumor suppression). (Bartuski, A J; et al. Genomics 43: 321-328, 1997). However, the serpin superfamily also includes members with the characteristic sequence that do not have serine protease-inhibitory activity. Several of these serpins have now been found to exhibit anti-angiogenic activity. These include PEDF, maspin, antithrombin III, and angiotensinogen.

PEDF (SerpinF1) is a member of the serpin superfamily by virtue of sequence and structural homology. PEDF is a 50 kDa protein having 418 amino acids, including a signal sequence having a predicted cleavage site between C₁₉/Q₂₀ as determine by analyzing the amino terminal region for the location of canonical cleavage sites. (program employed is available through the Center for Biological Analysis World Wide Web Prediction Server SignalP v2.0, Henrik Nielsen, Jacob Engelbrecht, Søren Brunak and Gunnar von Heijne: Identification of prokaryotic and eukaryotic signal peptides and prediction of their cleavage sites. Protein Engineering (1997) 10, 1-6).

PEDF is a natural, secreted, anti-angiogenic agent normally expressed in the eye (Dawson et al 1999, Science 285, 245-248). PEDF also suppresses ischemia-induced neovascularization of the retina mediated by VEGF. It inhibits neovascularization by inducing the apoptosis of endothelial cells that are forming new blood vessels (Stellmach et al 2001, Proc. Natl. Acad. Sci. USA 98, 2593-2597). PEDF is a survival factor for cerebellar granules and promotes survival and differentiation of spinal motor neurons. PEDF has recently been shown to be produced by differentiated ganglionic and Schwann cells within neuroblastomas and may be responsible for spontaneous tumor regression. (Crawford et al 2001, J. Cell Sci. 114, 4421-4428).

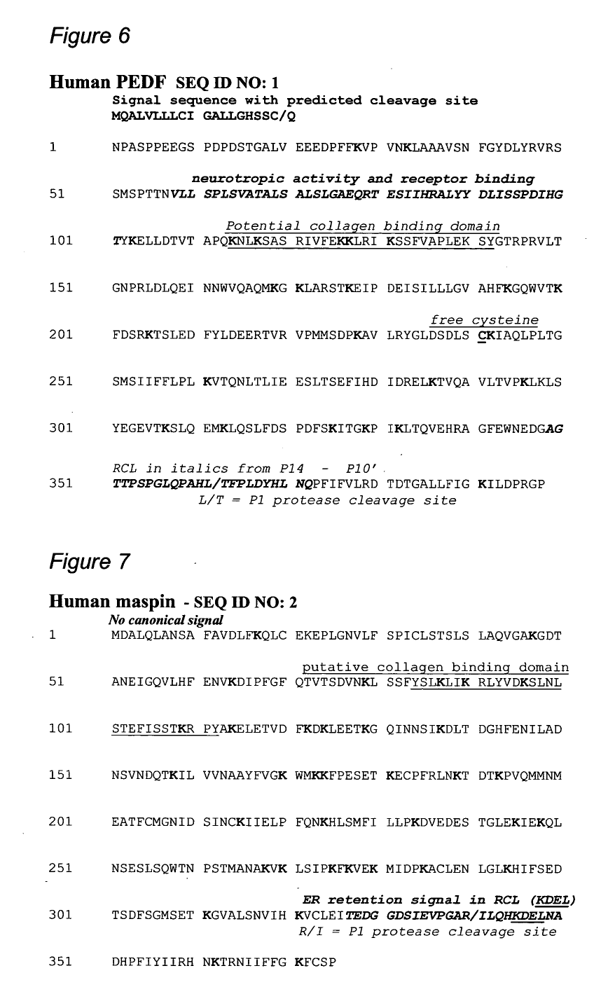

The amino acid sequence of human PEDF (SEQ ID NO: 1) including identification of certain functional domains is depicted in FIG. 6. As shown on FIG. 6, the receptor binding domain has been determined to be in a N-terminal portion located within amino acids V₇₈-T₁₂₁ of the full length sequence. This 44-mer fragment of PEDF exhibits neuronal differentiating activity on human retinoblastoma cells and is protective for motor neurons against chronic glutamate toxicity in organotypic spinal cord cultures. (Alberdi et al, J. Biol. Chem. (1999) 274 (44) 31605-31612).

The ovalbumin-type serpins (ov-serpins) are a subset within the serpin superfamily and are characterized by their high degree of homology to chicken ovalbumin, the lack of N- and C-terminal extensions, the absence of a signal peptide, and a serine rather than an asparagine residue at the penultimate position. At least seven members of the ov-serpin family had been mapped to a 300-kb region within chromosome 18q21.3/18q22: Maspin (PI5, a.k.a. serpinB5), squamous cell carcinoma antigen-1 (SCCA1) and -2 (SCCA2), plasminogen activator inhibitor type 2 (PAI2), cytoplasmic antiproteinase 2 (CAP2 or P18), bone marrow-associated serpin bomapin (PI10), and serpin B13 (headpin). Loss of heterozygocity at 18q has been identified with head and neck cancer and other malignancies. (Spring et al., Biochem Biophys Res Commun 1999 Oct. 14;264(1):299-304). Of the ov-serpins, maspin and headpin have anti-angiogenic activity.

Maspin (SerpinB5 or P15) has been shown to inhibit angiogenesis by blocking in vitro migration of endothelial cells and by in vivo inhibition of rat cornea neovascularization. (Zhang et al., Nature Medicine (2000) 6(2): 196). Maspin is expressed in normal mammary epithelial cells but not in most mammary carcinoma cell lines and is known to have tumor suppressor activity. (Zou, Z., et al. (1994) Science 263, 526-529). Surface-bound maspin is thought to be responsible for inhibition of cell invasion in mammary carcinoma. In addition to mammary epithelia, maspin is expressed in placenta, prostate, and small intestine. Maspin expression is down-regulated in breast tumor cells as demonstrated by both in vitro assay and by immunostaining of clinical specimens from breast cancer patients. Maspin expression has been reported to be consistently down-regulated during tumor progression. (Zhang and Zhang, Int J Oncol 2002 June; 20(6): 1145-50). Addition of maspin to tumor cells in vitro decreased the migration potential of breast and prostate tumor cells across a reconstituted basement membrane. (Sheng, S., et al (1994) J. Biol. Chem. 269, 30988-30993). Primary tumor growth and metastasis were significantly inhibited by maspin delivered as a gene therapy to mice bearing syngeneic mammary tumors. (Shi H Y et al., Mol Ther 2002 June;5(6):755-61).

Serpin B13, a.k.a PI13, hurpin and headpin, is expressed in normal oral mucosal tissue, skin, and cultured keratinocytes. PI13 is negatively regulated by UVB irradiation and was originally termed hurpin on this basis (From “HaCaT UV repressed”). (Abts H F, et al. J. Mol Biol 1999 Oct. 15;293(1):29-39). Down regulation of headpin mRNA expression has been demonstrated in squamous cell carcinoma of the oral cavity and in squamous cell carcinoma cell lines of the head and neck. (Spring et al., Biochem. Biophys. Res. Commun. 264: 299-304, 1999; Nakashima T, Biochim Biophys Acta 2000 Jul. 24;1492(2-3):441-6).

Antithrombin is a plasma protein of the serpin superfamily that may occur as several conformational variants. In its native form, antithrombin is a major regulator of blood clotting. Heat denaturation or cleavage of the carboxyl-terminal loop of antithrombin result in conformational changes that confer potent antiangiogenic properties to the protein. (O'Reilly M S, et al. Science 1999 Sep. 17;285(5435):1926-8; Larsson H, et al. Cancer Res 2000 Dec. 1;60(23):6723-9).

Angiotensinogen is a non-inhibitory serpin having renin as a substrate. Angiotensinogen is a 452 amino acid protein secreted primarily from the liver into the circulation. Cleavage of angiotensinogen by the aspartyl proteinase renin releases an inactive Ang I decapeptide and des(Ang I)angiotensinogen. (Célérier J. et al., J Biol Chem, (2000) 275 (14) 10648-10654). Anti-angiogenic activity is maintained in renin cleaved angiotensinogen (des(angiotensin I) angiotensinogen) and in reactive center loop-cleaved angiotensinogen, produced by selective and limited proteolysis by the protease V8. (Celerier J, et al. Hypertension 2002 February;39(2):224-8).

Although certain of the serpins have theoretical potential as clinically relevant anti-angiogenic agents, improvements are needed in their biological half-lives. The present invention provides compositions and methods for increasing the effective biological activity of anti-angiogenic serpins in vivo through covalent attachment of polymer molecules.

Covalent attachment of polyethylene glycol (PEG) and other polymers to certain proteins has resulted in improvements in pharmacological and some physiological properties (reviewed in Zalipsky & Lee (1992) Polyethylene Glycol Chemistry: Biotechnical and Biomedical Applications (ed. by J. M. Harris), Plenum, New York). However, the functional attributes of a protein are typically dependent on critical amino acid residues in the context of the native tertiary structure of the molecule. Covalent modification of these or other residues may result in destruction or blockage of active domains. (Veronese F. M. Biomaterials 22 (2001) 405-417). Thus, success in covalent polymer modification of one class of molecules does not predict the outcome with other classes even when using the same chemistry.

Polymer modification may employ any number of known and readily available polymers that generally contain at least one group that can be modified to form a moiety that is reactive with certain amino acid residues of the protein. The polymers that can be used are selected from the following that are readily available to those skilled in the art: Homo- and hetero-polymers, i.e., polymeric substances with repeating identical or non-identical subunits (homo-polymers and hetero-polymers, respectively) such as polyalkylene compounds such as for example polyalkylene oxides and glycols and their derivatives such as poly(oxymethylene), polyethyleneglycols and oxides and methoxypolyethyleneglycols and related homopolymers, such as polymethylethyleneglycol, polyhydroxypropyleneglycol, polypropyleneglycols and oxides, polymethylpropyleneglycol, and polyhydroxypropyleneoxide, which may be straight-chain or branched polymers (for example straight-chain polypropyleneglycols and branched chain polypropyleneglycols) and derivatives of the above including ethers, for instance of polyethyleneglycol or polypropyleneglycol such as the monomethyl ethers, monocetyl ethers, mono-n-butyl ethers, mono-t-butylethers and monooleyl ethers, esters of polyalkyleneglycols with carboxylic acids such as the monobutyl esters and monostearyl esters, and dehydration condensation products of the polyalkyleneglycols with amines such as propylamine and stearyl amine.

Other polyalkylene homo- and hetero-polymers that may be optionally employed include: a) polyvinyl compounds (such as poly(vinylpyrrolidone), polyvinyl alcohol, poly(vinyl acetate) and the copolymer poly(vinyl acetate-co-vinyl alcohol)); and b) polyalkylene polyols (such as polyoxyethylated glycerol, polyoxyethylated sorbitol (e.g polysorbates and polyoxyethylated glucose)).

Suitable polysaccharide homo and hetero polymers branched or unbranched polysaccharides, comprising saccharide monomers such as glucose, mannose, galactose, fucose, fructose, xylose, arabinose, glucuronic acid, sialic acid (neuraminic acid), galacturonic acid, mannuronic acid, D-glucosamine and galactosamine, which may be homopolysaccharides or heteropolysaccarides.

Block copolymers, i.e. copolymers formed by combining appropriate blocks of the above polymers and include for instance block co-polymers of small alkoxymonomers, e.g., polyethylene/polypropyleneglycol; block copolymers of ethylene and maleic anhydride; block copolymers of polyalkylene glycols and polyvinylpyrrolidone or polyvinyl alcohol; block copolymers of polyoxyethylene and polyoxy-propylene (e.g., poloxamers); block copolymers of the ethers, esters or dehydration condensation products of polymers of ethylene glycol and propylene glycol; and block copolymers of acrylamide and acrylic acid.

Preferred group of non-ionic block copolymers are the poloxamers. The term “poloxamer” means any di- or tri-block copolymer composed of the hydrophobe propylene oxide (POP, polyoxypropylene has the formula (C₃H₆O)ₓ and thus has a unit mw of 58) and the hydrophile ethylene oxide (POE, polyoxyethylene has the formula (C₂H₄O)ₓ and thus has a unit mw of 44). As used herein the term “poloxamer” includes the reverse poloxamers. PLURONIC® is a tradename for poloxamers manufactured by BASF. In Europe the tradename for pharmaceutical grade poloxamers manufactured by BASF is LUTROL. Poloxamers of the PLURONIC type are tri-block copolymers in which the propylene oxide block is sandwiched between two ethylene oxide blocks and has the following general formula and structure:

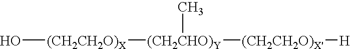

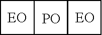

BASF poloxamers of the “reverse Pluronic®” type have the following structure:

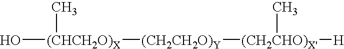

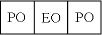

Depending upon the selected polymer, groups may already be available for reaction or it may be necessary to introduce groups by conventional techniques in a preliminary step. Generally these groups will be groups bonded to carbon atoms of the polymer. These groups will be selected such that they ultimately permit formation of the desired covalent link between the polymer and serpin. Preferred groups are primary (—CH₂OH) or secondary (—CHOH) alcoholic hydroxyls, for instance primary hydroxyls as found at the termini of polyalkylene glycols such as in PEG.

The use of capping groups may be an important feature of the reaction since this embodiment of the invention has special advantages. The function of the capping groups is to direct and control the site(s) of activation on the polymer. The capping group therefore must lack the properties of the reactive group and should preferably be an inert group with respect to reactivity with other molecules and should be of low toxicity. Suitable capping groups are well known to those skilled in the art, as are techniques for introducing such groups. A preferred capping group is methyl, an example of such is methylation of PEG resulting in a methoxy group at one end of a PEG molecule.

Protein PEGylation describes a form of polymer modification wherein one or more chains of polyethylene glycol (PEG) is covalently attached to a protein. PEG is a homopolymer having repeating ethylene subunits (OCH₂CH₂) of the formula:

PEG HO(OCH₂CH₂)ₙOH

A preferred polymer for use in accordance with the present invention is a PEG polymer HO—(CH₂CH₂O)ₓ—H where x is large enough for the molecular mass to be in the range of about 2,000 Da to about 40,000 Da. The size of the polymer actually used will be selected according to the desired properties of the end product.

In some cases, it is preferred that the PEG have only a single terminus available for derivatization with a reactive species such that crosslinking cannot occur. One such PEG is monomethoxy PEG (“MPEG”) having the following formula:

MPEG CH₃(OCH₂CH₂)ₙOH

Thus, a preferred polymer is MPEG (i.e. the polymer MeO-(CH₂CH₂O)ₓ—H which has one capping group, i.e. the methyl group and one reactive group, i.e. the hydroxyl group) where x is large enough to provide molecular weights of, for instance, about 2,000 Daltons (Da) to about 40,000 Da. In one embodiment, the MPEG is 20,000 Da (“20 kDa”). Is will be discussed, polymers of about 20,000 (i.e. a range of about 15 kDa to about 30 kDa) have advantages in controlling PEGylation through steric hindrance. Branched PEGs can also be used.

Since conventional polymerization techniques generally produce materials which are mixtures, in certain cases some purification may be required before the polymer is used in the process of the invention, for example, some preparations of MPEG have significant amounts of uncapped PEG, termed “high diol” preparations. Conversely, preparations having a low content of uncapped PEG are termed “low diol” preparations. Use of “high diol” polymers would lead to the production of bi-activated PEG and cross-linking on subsequent use and should be avoided for many applications. The amount of diol PEG can be measured using HPLC and removed via techniques know to one skilled in the art. For example, size fractionation or other means should therefore be used to reduce this PEG from the MPEG preparation, prior to activation. For example, the divalent PEG can be separated from the bulk MPEG using vesicle chromatography (Selisko et al (1993) J. Chromatogr. 641, 71-79).

A number of PEGylation chemistries are known to those of skill in the art. (reviewed in Roberts, M J. et al., Adv. Drug Del. Rev. 554 (2002) 459-476; Zalipsky S., Adv. Drug Del. Rev. 16 (1995) 157-182). Some examples of known techniques include:

Benzotriazole carbonate: U.S. Pat. No. 5,650,234 Dolence et al.

Cyanogen Bromide Method: U.S. Pat. No. 4,301,144 Iwashita & Ajisaka.

Cyanuric Chloride Methods: U.S. Pat. No. 4,179,337 Davis et al; Abuchowski et al (1977), J. Biol. Chem., 252, 3578-3586.

MPEG Propionaldehyde methods: U.S. Pat. No. 5,252,714 Harris and Herati.

Organic Sulphonyl Halide Methods: U.S. Pat. No. 4,415,665 Mosbach & Nilsson; Delgado et al (1990) Biotechnology and Applied Biochemistry, 12, 119-128; WO 90/04606 Delgado et al.

PEG-Aldehyde Methods: U.S. Pat. No. 4,002,531 Royer; U.S. Pat. No. 5,990,237 Bentley and Harris.

PEG-Maleimide and Related Methods: Goodson & Katre (1990) Biotechnology 8, 343-346.

PEG-Succinate Mixed Anhydride Methods: Ahlstedt et al (1983) Int. Arch. Allergy Appl. Immunol., 71,228-232; U.S. Pat. No. 4,261,973 Lee and Sehon.

Phenylalyoxal Method: EP-A-0 340 741 Maeda et al.

Phenylchloroformate Methods: Veronese et al (1985) Appl. Biochem. Biotechnol. 11, 141-152; WO 89/06546 Shadle et al.

Poly-PEG Maleic Acid Anhydride Method: Yoshimoto et al (1987) Biochemical and Biophysical Research Communications, 148, 876-882.

Succinimidyl Active Ester Methods: Abuchowski et al (1984) Cancer Biochem. Biophys. 7, 175-186; U.S. Pat. No. 4,412,989 Iwashita et al; Katre et al (1987) Proc. Natl. Acad. Sci. USA, 84, 1487-1491.

Succinimidyl Carbonate Method: WO 90/13540 Zalipsky; T. Miron and M. Wilchek, Bioconjug. Chem. 4 (1993), pp. 568-569.

Urethane linkage methods including use of carbonylimidazole: Beauchamp et al (1983) Analytical Biochemistry, 131, 25-33; p-nitrophenyl carbonate and trichlorophenyl carbonate: Veronese et al., Appl. Biochem. Biotechnol. 11 (1985) 141-152.

From a pharmaceutical perspective, the primary benefit of polymer modification is a substantial reduction in systemic clearance. Polymer modification, for example by PEGylation, increases the hydrodynamic radius of a protein, which reduces renal clearance. PEGylation may also reduce clearance via receptor- or cell-mediated processes, or by reducing proteolysis. As a result of these effects, PEGylated proteins typically have much longer circulating half-lives than their unmodified counterparts, often by factors of 5- to 100-fold or more. Proteins, in particular labile proteins, such as many of the serpins would require frequent administration in order to realize their full potential. The anti-angiogenic potential of PEDF and maspin for example would be lost when the protein is below the therapeutic threshold. In contrast, polymer modification potentially provides sustained protein presence in the therapeutic range.

Protein PEGylation can also offer other benefits that may be of substantial value in specific situations. PEGylation has been shown to reduce both the immunogenicity (tendency to elicit antibody formation) and the antigenicity (ability to bind and be neutralized by antibodies) of some proteins. Other benefits may include improved solubility, reduced protein aggregation, improved bioavailability after subcutaneous injection, and improved thermal and mechanical stability.

Extended half-life generally correlates with both the number and size of the attached PEG chains. Thus, a single 20 kDa PEG chain will typically have greater impact than a 5 kDa chain. Similarly, multiple 5 kDa chains will have greater impact on biological half-life than just one. However, each additional PEG chain may result in a concomitant loss of intrinsic activity. (Clark et al. J. Biol. Chem. (1996) 271: 21969-77). Thus, the primary drawback of PEGylation is reduction in protein specific activity. This can have multiple causes. PEGylation of an active site residue can directly block activity. PEGylation at other sites may sterically hinder interactions with receptors or substrate molecules or cause conformational changes, even if the active site is not directly modified.

As a consequence of these phenomena, polymer modification does not necessarily provide an advantage and may be expected to have fewer advantages with larger proteins such as the serpins in which rapid elimination on the basis of size might not be expected. Furthermore, the serpins have a multidomain structure and the relative contribution of the various domains to recognized disparate effects are not understood. Despite considerable practical experience with PEGylation of various proteins, a benefit to polymer modification has not hereto for been taught or suggested for the serpins.

### EXAMPLE 1

### System for Assessing Anti-Tumor Effects

SK-N-AS cells were used for experiments to test PEDF protein. SK-N-AS cells may be obtained from the American Type Culture Collection (ATCC; Rockville, Md., US). This cell line is a human neuroblastoma cell line that has been previously used by others for testing angiogenesis inhibitors (Klement G et al, J Clin Invest 2000 April;105(8):R15-24. For all protein studies, SK-N-AS tumors were implanted in 6-8 week old, female, CB17 SCID mice purchased from Charles River. For protein studies, 1×10⁶ cells were subcutaneously implanted in a volume of 200 microliters. Treatment was initiated when a palpable tumor was present (generally 5-8 days post treatment). Utilizing this human cell line in a xenograft model involving immunodeficient mice obviated potential confounding effects of a host immune response against recombinant hPEDF protein.

Differences in tumor growth rate between treatment groups were assessed by repeated measures ANOVA. When a significant (p<0.05) treatment versus time interaction was present, differences between individual groups means were assessed by Duncan's test. Differences between individual group means were assessed at the 0.05 and 0.10 level of type I error.

### EXAMPLE 2

### Generation and Purification of PEDF Protein

A PEDF-His tag fusion protein was produced from a stably transfected cell line (HEK-SLED cells; Dr. Noel Bouck, Northwestern University). The cell line was maintained in DMEM 10% FBS. For the production of culture supernatant for protein purification, exponentially growing, ˜70% confluent T150 flasks were washed 2× with 40 ml PBS and incubated with serum-free DMEM for a period of 48-72 h. Culture supernatants were collected at the end of the incubation and cell debris were removed by centrifugation. Typically, 300 ml of supernatants for purification were collected from 30 T150 flasks. Alternatively purified PEDF protein can be obtained from BioProducts Maryland, Inc., Middletown, Md., US.

A commercially available kit was utilized for purification of the His-tagged protein based on nickel affinity column chromatography (INVITROGEN PRO-BOND Resin and System) using native conditions. Essentially, 300 ml culture supernatant was collected and phenyl methyl sulfonyl chloride (PMSF stock: 10 mg/ml) added at 0.5 ml for every 45 ml of supemate. The supemate was concentrated to about 15 ml and an equal volume of Native Binding (NB) buffer pH 7.46 was added and concentrated back to ˜15 ml. The process was repeated several times and a total volume of 50-60 ml of NB was used with the supernate finally concentrated to 4 ml. To this, 3 ml of NB pH 7.6 was added and in addition to 11 microliters of the above mentioned PMSF stock. The column was prepared by washing with dH₂O (2×) and with NB buffer pH 7.46 containing 50 mM imidazole (3×). The supernate was clarified by centrifugation (fraction #1) at 3000 rpm for 10 minutes and the clarified supemate was loaded on column and incubated for 60 h at 4° C. on a spinner. The column was washed with 3× with NB pH 7.46, and 3× with Native Wash (NW) Buffer pH 6.0. The column was then washed washed 2× with 50 mM imidazole and 2× with 100 mM imidazole. PEDF was eluted with final 200 mM and 500 mM imidazole washes and these fractions were extensively dialyzed against PBS to remove the imidazole.

Other methods may be employed for purification of PEDF including immunoaffinity chromatography using poly or monoclonal antibodies specific for PEDF.

### EXAMPLE 3

### Local Peritumoral Delivery of Unmodified PEDF

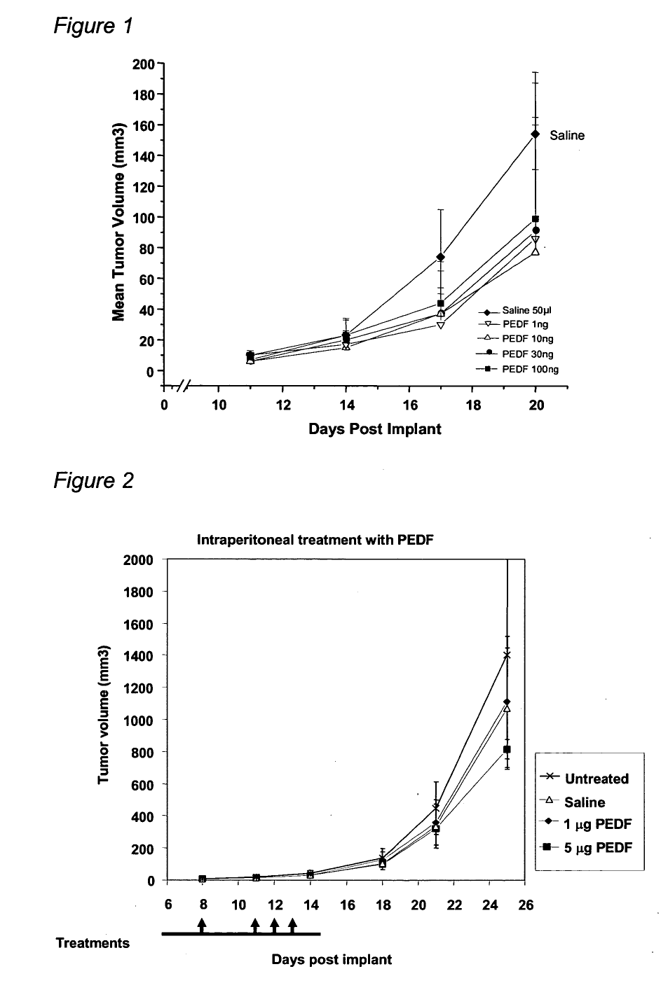

Groups of 10 animals each, bearing 5 day old, subcutaneously implanted SK-N-AS tumors, were treated peritumorally, 5 times/week with 1, 10, 30, 100 nanograms of PEDF protein in 50 microliters saline, or with 50 microliters saline alone. Tumors were measured at regular intervals and data plotted as mean±S.D. for each time point. As shown in FIG. 1, essentially all treatment doses were equally efficacious in inhibiting tumor growth when compared with saline, suggesting that even the lowest treatment dose of 1 nanogram was biologically efficacious for local peri-tumoral delivery in this model.

### EXAMPLE 4

### Systemic Delivery of Unmodified PEDF

Groups of 6 animals each, bearing 8 day old, subcutaneously implanted SK-N-AS tumors, were treated i.p. on days 8, 11, 12, and 13 with 1 or 5 microgram doses of PEDF protein in 200 microliters saline, or 200 microliters saline alone. Tumors were measured at regular intervals and data plotted as mean±S.D. for each time point. The results are shown in FIG. 2. Although the 1 microgram dose was ineffective, the 5 microgram dose showed a reduction in tumor size at the last time point (25 days post implant).

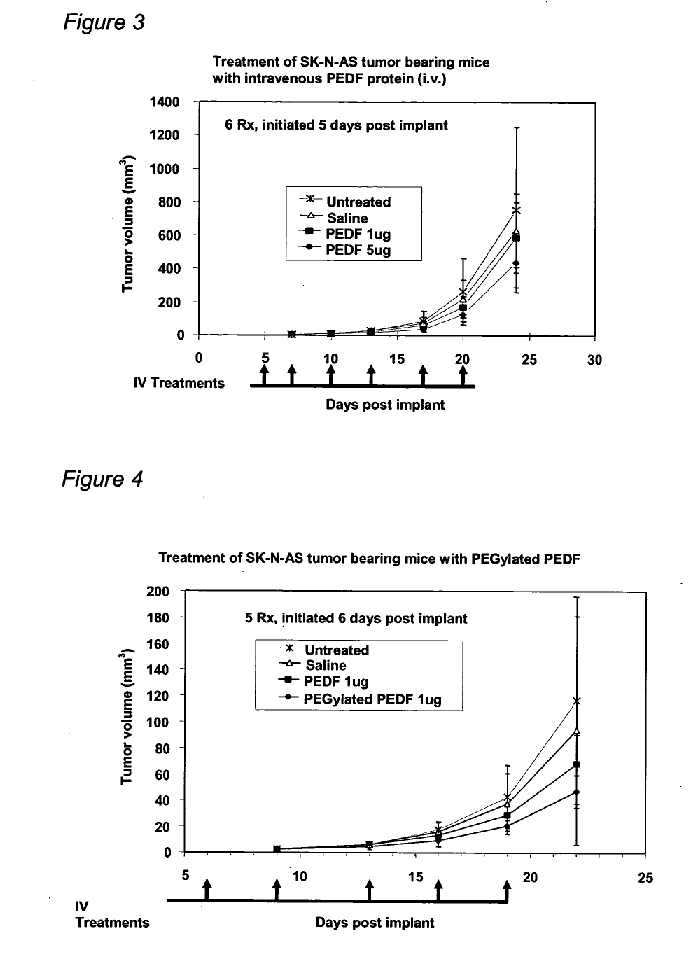

Groups of 6 animals each, bearing 5 day old subcutaneously implanted SK-N-AS tumors, were treated i.v. twice a week for a total 6 treatments with 1 or 5 microgram doses of PEDF protein in 75 microliters saline, or 75 microliters saline alone, or left untreated. Tumors were measured at regular intervals and data plotted as mean±S.D. for each time point. As shown in FIG. 3, the 1 microgram dose behaved very similar to the saline treatment and apparent efficacy in the form of tumor growth inhibition throughout the study was observed only with the 5 microgram dose. Having determined that PEDF protein was efficacious as an anti-angiogenic factor in tumors, PEGylation was tested to determine if the efficacy could be improved.

### EXAMPLE 5

### Polymer Modification for Improved Anti-Tumor Activity of Serpins

Serpins are either substrates or inhibitors for proteases and are thus sensitive to proteolytic degradation. PEDF and maspin bind collagen and thus could be expected to have restricted ability to move through the circulation. For these reasons, the present inventors undertook to improve the biological activity of the anti-angiogenic serpins by polymer modification and are the first to disclose use of PEGylated anti-angiogenic serpins for use in improving the anti-tumor effects of these proteins.

PEDF does not inhibit proteases and does not require the serpin reactive center loop for neurotropic activity. (Becerra et al. J. Biol. Chem. (1995) 270(43) 25992-9). Unlike the inhibitory serpins, PEDF does not undergo a profound conformational change upon cleavage of the reactive center loop. FIG. 6 shows the amino acid sequence of PEDF (NCBI GenBank Accession Number P36955, GI:201788323). The PEDF RCL sequence from P14-P10′ is indicated in FIG. 6. (Celerier J. et al., J. Biol. Chem. (2000) 275(14) 10648-10654). The central portion of the RCL is highly conserved between human, murine and bovine PEDF. Furthermore, the reactive center loop is not required for anti-angiogenic activity. Thus cleavage with trypsin at H₃₈₁//L₃₈₂ of the full length sequence generates a N₂₁-L₃₈₂ PEDF fragment that retains anti-angiogenic activity. (Bouck et al. U.S. Pat. No. 6,288,024). PEDF binds preferentially to collagens type I and III, but not to laminin, fibronectin, gelatin or type II or IV collagens in vitro. (Kozaki et al., J. Biol. Chem. (1998) 273(24): 15125-15130). Maspin also preferentially binds type I and III collagens. Analogy and alignment to the putative collagen binding domain of maspin places a potential collagen binding domain in the PEDF mature sequence in the region from amino acids K₁₁₄-Y₁₄₂ as shown on FIG. 6.

Maspin is a 375 amino acid non-inhibitory ov-serpin having tumor suppressing activity. (Sager et al., U.S. Pat. No. 5,905,023) Maspin is a substrate for single chain tissue plasminogen activator. (Sheng et al. PNAS (1998) 95:499-504). Maspin lacks a canonical signal sequence but does have a weakly hydrophobic amino terminal domain that appears to function as a weak secretion signal. Furthermore, the maspin sequence (SEQ ID NO: 2), depicted in FIG. 7 (consistent with NCBI Accession number P36952) reveals the presence of a canonical ER retention domain, KDEL₃₄₅₋₃₄₈, in the C terminal reactive center loop. Presumably as a combined consequence, maspin is predominantly an intracellular cytosolic protein, but may associate with secretory vesicles resulting in cell surface expression. (Pemberton et al., J. Histochem & Cytochem (1997) 45(12): 1697-1706).

Maspin has been reported to be an unstable serpin having extreme sensitivity to limited proteolysis at its reactive site loop. (Fitzpatrick et al., Protein Eng (1996) 9(7): 585-9). Although the RCL is necessary for inhibition of cell invasion, inhibition of angiogenesis is independent of reactive site loop integrity (Zhang, M. et al. (2000) Nat. Med. 6, 196-199.). Maspin interacts with collagen and preferentially binds to collagen subtypes I and III, presumably by virtue of a collagen binding domain thought to be located in the region of amino acids Y₈₄-Y₁₁₂, indicated on FIG. 7. (Blacque O E and Worrall D M. J Biol Chem 2002 Mar. 29;277(13):10783-8).

In alternate embodiments, the anti-angiogenic agents to be polymer modified are fragments of anti-angiogenic serpins that retain anti-angiogenic activity but lack other domains. For example, a recombinant maspin may be employed in which molecular techniques known to those of skill in the art are used to add a signal sequence or replace certain of the amino terminal amino acids. In addition, recombinant techniques can be employed to generate a recombinant maspin in which carboxy terminal sequences that may cause retention of maspin in the ER are removed. In this way the ability to generate recombinant maspin in sufficient quantities for use in vivo may be obtained. Further carboxy terminal sequences may be removed up to and including the RCL in order to increase the protease resistance of the protein.

### EXAMPLE 6

### PEGylation Considerations

Protein Considerations:

The quality of the protein preparation can have a major impact on the PEGylation reaction. The protein should be as free of contamination as possible. If the protein tends to stick to containers, and under normal circumstances is formulated with carrier protein, careful consideration will need to be given to alternative carriers. Carriers capable of reacting with activated PEG should be avoided.

Protein fragmentation, in particular, should be avoided. This introduces a variable and unknown number of amino termini that compete for the activated PEG. Buffers containing nucleophiles should be avoided and the buffers should have sufficient buffering capacity. The tendency of the protein to precipitate and aggregate is also important. PEG induces precipitation and/or aggregation quite markedly in some proteins. Protein and PEG concentrations both influence this.

PEGylation Matrix:

To identify the best candidate PEGylated product, a PEGylation matrix may be employed subjecting the target protein to varying degrees of PEGylation with PEGs of different chain lengths. This can be achieved by varying the concentrations of either activated PEG or protein, the total reaction time, the pH and other components/parameters which effect protein structure/solubility.

In general the retention of bioactivity does vary with the number of PEG chains attached, tending to diminish at higher PEG chain densities. However, it should be noted that this may not necessarily be due to the number of PEG chains attached per se, but rather due to the coupling conditions and either the more prolonged PEGylation time or the increased concentration of activated polymer required to achieve high degrees of PEGylation.

In general there is a trend of increasing circulation time with increasing PEG chain lengths. However, candidate products with different chain lengths need to be assessed in efficacy studies in vivo. A high PEG load can significantly alter biodistribution and can even exclude the product from normal tissues. Thus, the optimum chain length and degree of substitution will vary from protein to protein and depend on the location of the target cells of the biologically active PEGylated agent. The optimum number of PEG chains for maximum biological activity of PEDF and maspin will be determined empirically according to the PEGylation matrix.

Most PEG chemistries result in attachment of PEG to the most reactive groups, in order of reactivity, thiol>alpha amino group>epsilon amino groups of lysine residues. The imidazole group of histidines may react under special conditions including certain pH ranges. Unprotonated amino residues are reactive, while the protonated forms are not. Thus depending on the pH of the reaction and the pKa of the amino group, the alpha amino and epsilon amino groups of lysine are considered the primary sites for PEGylation on proteins. Free thiols are particularly reactive but are rare in most proteins. PEDF is unusual in having a free thiol at C₂₄₁. This residue is highlighted on FIG. 6.

In FIGS. 6 and 7 the lysine residues (K) of PEDF and maspin are shown in bold letters. In the tertiary structure, a number of these lysines will be buried and will not be accessible to PEGylation. Furthermore, where the PEGylation reaction is conducted at a pH of ˜6.5 to ˜7, as is possible with certain PEGylation chemistries, such as for example tresyl PEGylation and succinimidyl carbonate PEGylation, it is possible to limit which lysine residues are PEGylated. This is because the pKa of the ε-amino group on lysine residues is usually above pH 9, making most of these amino groups protonated and unreactive at pH 7. Since the α amino group of the N-terminal amino acid has a pKa around 6, it will mostly be unprotonated, a better nucleophile and will be more selectively PEGylated. It is expected that with PEGylation employing larger PEG moieties, such as 20 kDa PEG, that after attachment of the first PEG, steric inhibition will deter PEGylation of other nearby lysines or other reactive residues. This phenomenon will be more pronounced with each successful PEG molecule that attaches. As a result, an advantage of PEGylation with longer PEGs is that PEGylation of other residues, with concomitant loss of activity, is minimized, even as the reaction is prolonged to maximize the number of protein molecules with at least one PEG attached. In contrast, with smaller PEG moieties such as 5 kDa PEG, running the reaction under conditions that maximize that each protein molecule will have at least one PEG moiety attached, is likely to result in more, different residues being PEGylated and thereby increasing the possibility of inactivating that particular protein molecule.

Bioassay of PEGylated Products:

Estimation of the relative retention of biological activity of the PEGylated products is first performed in whole reaction mixtures. The bioactivity of individual PEGₙ-protein species is then confirmed after fractionation of the reaction mixture, usually by chromatography.

For regulatory purposes it may be important to establish over what range of values of “n” the protein remains biologically active and to characterize which residues are modified. For GM-CSF, for example, “n” values between 1 and 3 lead to no significant reduction in colony-stimulating activity and may therefore all be included in the product.

Most PEGylated products to date used in the clinic, including PEG-IL-2, ADAGEN™ and ONCOSPAR™, have been statistical mixtures of PEGylated products.

### EXAMPLE 7

### TMPEGylation Parameters

In one embodiment, PEDF protein was PEGylated using tresylated monomethoxy polyethylene glycol (TMPEG). TMPEG has the formula:

TMPEG CH₃(OCH₂CH₂)ₙOSO₂CH₂CF₃

PEGylation with TMPEG is a one-step reaction in which the PEG molecule is directly attached to the protein of interest with liberation of the activating tresyl moiety. A typical reaction is shown below:

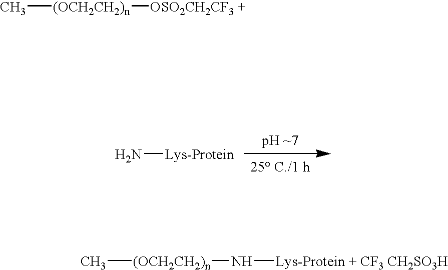

Under proper conditions, TMPEG readily reacts with primary amines to form a covalent linkage between the amine group and the PEG. In a protein, these primary amines correspond to the amino terminus and the side chains of lysine residues. The tresyl group is displaced in this reaction, as the sulfonyl group is more sterically hindered, resulting in a stable secondary amine linkage of the PEG to the protein. An advantage of PEGylation with TMPEG is that it does not alter the number of charged groups in the protein. As the TMPEG couples to the primary amines of lysine residues and the amino terminus, these primary amines are converted to secondary amines, which retain their positive charge. In contrast, other PEGylation chemistries that target lysines and the N-terminal residue may convert the positively charged primary amines to neutral amides resulting in alteration of surface charge. The significance of this effect depends on the protein and may be assessed empirically. TMPEG coupling reactions are performed under mild conditions physiological pH, which may minimize losses in protein activity due to denaturation or deamidation relative to other chemistries that require harsher coupling conditions and/or non-physiologic pH.

TMPEG used for coupling reactions is prepared by derivatization of the free hydroxyl group of MPEG, USP-grade manufactured by Nippon Oil and Fats (NOF Corporation, Japan). These MPEGs contain only small percentages of diol.

A suitable TMPEG has been prepared according to the following general method: MPEG is dried by azeotropic distillation with toluene then reacted with tresyl chloride, at a ratio of 2.5 moles of tresyl chloride per mole of available —OH groups on the MPEG, in dichloromethane using pyridine as a base catalyst. The polymer is recovered by removal of the dichloromethane in vacuo. The excess tresyl chloride and pyridine are then removed by precipitation from methanol/HCl solution in the cold twelve times, centrifugation being the method of recovery. The TMPEG product is washed with methanol, concentrated in vacuo, the solid lyophilized, dispensed into amber glass vials, and stored under nitrogen. The material is stored at 2-8° C. in the dark.

For an example of a suitable manufacturing process:

low diol MPEG is dissolved in dry toluene at a ratio of 2.2 ml toluene per gram of MPEG and any residual water is removed as an azeotrope with toluene by rotary evaporation at reduced pressure. In this procedure, after the bulk of toluene is removed by rotary evaporation at ˜40° C., the preparation is heated to 70° C. and the rotary evaporation continued. Alternatively, the MPEG is dried with azeotrope distillation using benzene by distillation of the water-benzene azeotrope and then the benzene;

dry dichloromethane (DCM) is prepared. For example, on a laboratory scale the DCM can be dried over 3 angstrom molecular sieves, at a ratio of 1 gram per 5 ml of solvent, overnight at room temperature;

the water-free MPEG is then immediately dissolved in the dry DCM at a ratio of 2.2 ml DCM per gram of MPEG starting material;

the MPEG/dichloromethane mixture is cooled to less than about 2 [symbol] ;

dry pyridine, precooled on ice, is added dropwise with constant stirring at a ratio of about 3.4 moles of pyridine per mole of available —OH groups on the MPEG. Thus, in the case of 20K MPEG, ˜0.014 ml pyridine is added per gram of starting material. Immediately following the pyridine, dry tresyl chloride (Fluka), precooled on ice, is added dropwise with stirring at a ratio of 2.5 moles of tresyl chloride per mole of available —OH groups on the MPEG. Thus, in the case of 20K MPEG, ˜0.014 ml of tresyl chloride is added per gram of starting material. The reaction is continued for 2 hour at room temperature with constant stirring;

the dichloromethane is removed by evaporation under reduced pressure without heat, leaving a solid (TMPEG);

residual pyridine is removed from the solid by redissolving the solid in a methanol:hydrochloric acid mixture (1000:0.3 v/v), or alternatively ethanol:hydrochloric acid, and then precipitating overnight at reduced temperature (−20° C.). The precipitate is recovered, for example by centrifugation (at 1,100×g, for 10 minutes) at 0° C., and the supernatant checked for pyridine content by measuring the absorbance at 255 nm. This procedure is repeated until no pyridine can be detected, usually 12 to 15 washes;

finally, the precipitate is washed with dry methanol or ethanol and the methanol or ethanol removed by evaporation under reduced pressure, preferably at least twice;

the TMPEG is finally dried by freezing in liquid nitrogen and lyophilizing under reduced pressure.

The hydroxyl content of the white solid as determined by ¹H NMR obtained is undetectable, suggesting the majority of the MPEG is now TMPEG. The actual amounts of residual MPEG, MPEG-Cl and other impurities are assessed by rpHPLC.

The TMPEG is tested for identity and purity in three primary physio-chemical assays, namely, ¹H-NMR, elemental analysis and reverse phase chromatography. The process used to manufacture this TMPEG was optimized using biological assays to measure the impact of trace impurities on biological activity.

Analysis of TMPEG by ¹H-NMR: This assay is used to identify and assess the purity of TMPEG by it proton NMR. The ¹H-NMR spectra can establish the presence of the tresyl group, approximate degree of tresylation and extent of contamination. The ¹H-NMR analysis is performed in dry CDCl₃. The degree of tresylation is determined by comparing the ratio of the tresylated multiplet and quartet signals to the methoxy signal.

Elemental Analysis of TMPEG: For elemental analysis, the percent F and S atoms are used to estimate the degree of tresylation based on the average molecular weight of the parent MPEG. Expected values for 20 kDa TMPEG are 54% C; 9.1% H; 0.28% F; 0.16% S; low C1.

Reverse Phase HPLC Analysis: Reverse phase chromatography establishes the identity of the TMPEG raw material based on retention time and the relative purity of the material based on the presence and area under the curve (“AUC”) of impurity peaks. TMPEG is well resolved from the parent MPEG, on a reverse phase system using a PLRP-S 100 Å 5micron 150×4.6 mm column from Polymer Laboratories (UK) and a gradient of acetonitrile in water from 30% to 50% over 20 minutes followed by 50% acetonitrile to 100% acetonitrile over 5 minutes. The TMPEG and various impurities are detected using an evaporative light scattering detector connected at the outlet. Identification of the TMPEG and MPEG peaks by means of spiking with known materials is recommended. Acceptable lots have >83% AUC and retention times consistent with standard. Some TMPEG preparations also contain a minor contaminant (MPEG-C1) that runs between MPEG and TMPEG. This contaminant normally does not exceed 5% of the preparation. MPEG values are typically less than 15% and MPEG-C1 levels are typically less than 4%. There are no adverse consequences for MPEG-C1.

Further chemical characterization assays that were conducted include: pH of a TMPEG solution to test for amount of free tresyl acid; water content of the TMPEG and break down of the PEG ether linkages by measuring A₃₂₀ of a TMPEG solution.

pH of a TMPEG solution: This assay determines the pH of a TMPEG solution in water. Low pH samples may be indicative of degradation due to hydrolysis to form the acidic tresyl acid by-product. Required values are 4+/−0.2 pH units for a 2 mg/ml aqueous solution.

Water Content of TMPEG by Karl Fischer titration: This method determines the amount of water present in a lyophilized TMPEG sample by coulimetric titration. Required values are <0.2% for a 2 mg/ml non-aqueous solution.

A₃₂₀ of a TMPEG solution: This method indirectly tests for the presence of oxidation products based on a sample's absorbance at 320 nm. Unusually high values could indicate an oxidative process has occurred. Required values are <0.1 AU.

Optionally the viscosity of a TMPEG solution in water may be determined although not as sensitive as A₃₂₀ to the presence of oxidation products. Viscosity values of 4.1-4.4 cp for a 2 mg/ml aq. solution are typical. Average Mw and polydispersity by SEC was determined to be +/−5% Mw deviation with a polydispersity ≦1.05%.

In addition, the process described above was developed for determining that a given manufacturing process produced suitable material by analyzing initial lots for any adverse impact on the biological activity in several reporter systems due to trace impurities. This was deemed necessary as TMPEGs produced by different manufacturers, which rendered products with similar chemical and physical properties, had varied performance towards conservation of bioactivity when used to PEGylate proteins. The process detects otherwise unidentified toxic materials and other species that affect the rate of PEGylation. A number of biological analyses have been identified that can be used to discriminate “good” and “bad” processes for manufacturing TMPEG. Typically, conventional physical and chemical characterization of TMPEG are insufficient to discriminate between “acceptable” and “unacceptable” processes for manufacturing the polymer. As trace impurities based on a weight per weight percentage basis seem insignificant, they are more than stoichiometric when compared to the concentration of the residues in the protein being measured. For example 0.2% water is actually a two fold molar excess to the TMPEG, and while the TMPEG used in PEGylation is in the millimeter concentration range, the lysine residue on a typical protein is ˜1,000 fold lower, in the micrometer range. Although meeting any or all of these parameters may not be necessary under certain circumstances and further discriminating tests may be developed, they have been found to be useful parameters for obtaining a consistent product.

Thus, the following assays have been used to optimize the manufacturing process of the TMPEGs to ensure optimum function of PEGylated products: conservation of biological activity of PEG-GM-CSF, toxicity and conservation of bioactivity using a bioassay for PEGylated erythropoietin (PEG-EPO), and rate of reactivity in a standard coupling reaction with a standard protein such as lysozyme.

GM-CSF Activity Bioassay: This bioassay determines the remaining biological activity of a PEG-GM-CSF sample after a TMPEG coupling reaction. The assay is based on the GM-CSF-induced cell proliferation. For example, cultured TF-1 cells, which proliferate in response to GM-CSF, are exposed to increasing double dilutions of the GM-CSF samples (from 0.0029 ng/ml to 0.75 ng/ml) and their proliferation is monitored by the oxidation of WTS-1 dye (Boehringer). The growing cells metabolize the dye into a yellow-brown product in mitochondria. The color intensity of the dye product is directly proportional to the growth rate of the cells. Low degrees of retention of bioactivity (proliferation) are indicative of unsuitable TMPEG processes, possibly due to the presence of impurities or problems with the chemistry. Required values are >80% retained bioactivity.

EPO Toxicity Bioassay: This bioassay examines the effect of TMPEG impurity-related toxicity after a sample of EPO has been PEGylated with TMPEG. Cultured TF-1 cells are exposed to increasing double dilutions of the EPO (from a maximum of 10 U/ml) and their proliferation is monitored. The aim of this assay is to establish the Inhibitory Concentration 50 (IC₅₀) for activated PEG derivatives using a proliferation assay with EPO. This is achieved by comparing the dose-response curves of the different samples.

Cultured cells are exposed to increasing double dilutions of the EPO (from 10 U/ml) and their proliferation is monitored by ³H-Thymidine uptake. The growing cells incorporate ³H-Thymidine in their DNA and the quantification of the uptake is established using a β-counter and is proportional to the toxicity of TMPEG samples.

Alternatively, proliferation can be measured by the metabolism of cell proliferation reagent WTS-1 (tetrazolium). The growing cells metabolize WTS-1 into a yellowish brown product in mitochondria. The color intensity of the dye product is directly proportional to the growth rate of the cells. Required values are inhibitory concentrations (IC₅₀)>8 U/ml.

Rate of Reactivity with Lysozyme (CE Analysis): This assay uses capillary electrophoresis to monitor a reaction between lysozyme and TMPEG. The proportion of remaining unmodified lysozyme after a 21 minute reaction with TMPEG gives a relative estimate of reactivity. Values in the range of 35-45% were obtained for the TMPEG used to PEGylate PEDF.

Care of the TMPEG is a very important issue given the hygroscopic nature of PEG. TMPEG, for example, can capture water from silica gel. The small amount of TMPEG and corresponding sham MPEG for research studies are typically stored in the dark at 4° C. in a desiccator over P₂O₅ and under nitrogen atmosphere (oxygen free and with water content of less than 1 ppm). Once opening a TMPEG sample, proper care should be taken (i.e., minimize exposure time to air) to keep the TMPEG samples as dry as possible, if it is not used right away.

### EXAMPLE 8

### TMPEGylation Parameters

In most cases the TMPEG reaction works best when carried out at pH 7 in any non-nucleophilic buffer, such as phosphate. Care must be taken to insure that the buffering capacity is sufficient to maintain a constant pH throughout the course of the reaction, (keeping in mind that tresyl acid is liberated and will lower the pH if the buffer is insufficient). The reaction will proceed slower at pH values below 7, and as the pH drops below 6, most of the nucleophilic amines are significantly below their pKa and thus unreactive. At higher pH values the reaction proceeds more rapidly, however, the chance of a product with an alternative, less stable, linkage (protein amine coupled via an elimination mechanism rather than tresyl displacement) increases.

The reaction proceeds within an hour at room temperature, however, when protein stability or more selectivity is desired, the reaction can be carried out at lower temperatures with the caveat being that the reaction now usually runs longer, ˜16 hours.

In general a large molar excess of TMPEG to protein ensures efficient PEGylation of most proteins. However, larger excesses also lead to other different residues being PEGylated and decreased activity. More selective PEGylation occurs at low or stoichiometric ratios of TMPEG to protein, at the expense of yield. The appropriate molar ratio may be determined empirically for each protein.

At pH 7, the TMPEG reacts with water at a half-life of ˜2.5 hours. At higher pH values it is even shorter making it unnecessary to stop or quench the reaction to retain bioactivity by preventing other residues from slowly reacting with the remaining TMPEG. In some cases, after running a time course and looking at which residues are modified and the effect on bioactivity, it may be necessary to quench the reaction. Termination of the reaction can be obtained simply by lowering the pH, such that the amino groups are all protonated and now unreactive. Quenching of the TMPEG reaction has been shown to take place by dropping the reaction pH below 5. This is accomplished by the addition of an acidic buffer such as sodium acetate. This is a viable method, especially if one considers that purification via ion exchange chromatography can also be carried out at pH 4.5, thus ensuring that the TMPEG reaction remains ‘quenched’ until removed.

Alternatively, the reaction can be terminated by addition of an excess of compounds having nucleophilic groups that are reactive with TMPEG. For example, TMPEGs that react with primary amines can be quenched an amino acids such as arginine, histidine or lysine.

The physico-chemical characterization and biological activities of PEG-proteins can be performed in many cases without removal of the excess PEG present in the sample, provided that control samples are included to account for any interference due to the uncoupled polymer. If removal of the excess unreacted PEG is desired, a number of means known to those of skill in the art can be employed. For example, PEG does not interact with either cationic or anionic exchangers, and therefore, ion exchange chromatography is suitable to remove the excess PEG when the PEGylated conjugates bind to the column. Ion exchange chromatography has been applied to an increasing number of protein and virus preparations and may be a preferred method. The starting conditions are always those already applied to the unmodified target material. The PEGylated conjugates show, in general, reduced interaction with the matrix and therefore elute earlier than the unmodified material.

### EXAMPLE 9

### Increased Biological Activity of PEDF Obtained by Polymer Modification

A vial of TMPEG-20 kDa (Low diol TMPEG 20k, NOF Corporation) that had met performance criteria was removed from a desiccator and allowed to reach room temperature. 100 milligrams of TMPEG-20 kDa was transferred to an EPPENDORF tube. PBS (200 microliters) was added to the tube followed by vortex mixing until in solution to get a 500 mg/ml stock. 32.5 microliters of TMPEG stock was added to 500 microliters of PEDF (965 micrograms/ml protein) in an EPPENDORF tube. The tube was wrapped in parafilm and floated in a circulating water bath at 25° C. for 90 minutes. The TMPEGylated protein was divided into aliquots in freezing vials and stored over liquid nitrogen until further use.

### EXAMPLE 10

### Intra-Venous Treatment with Pegylated PEDF Protein

Groups of 6/7 animals each, bearing 6 day old, subcutaneously implanted SK-N-AS tumors, were treated i.v. twice a week for a total 5 treatments with 1 microgram doses of native or PEGylated PEDF protein in 75 microliters saline, or 75 microliters saline alone, or left untreated. Tumors were measured by calipers for length, width and depth such that a volume determination could be made. Measurements were performed at regular intervals and data plotted as mean±S.D. for each time point.

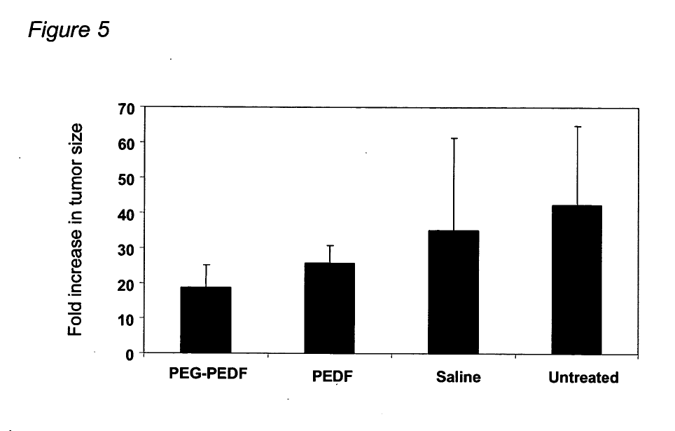

As shown in FIG. 4, the 1 microgram dose of PEGylated PEDF resulted in an improved inhibition of tumor growth as compared with unPEGylated PEDF. When evaluated in terms of tumor size progression in individual animals as shown in FIG. 5, the difference between PEGylated PEDF and untreated animals at the last time point was statistically significant, *p=<0.03 (Students two-tailed t-test). In contrast the difference between native PEDF and untreated animals at the last time point was not statistically significant, p=0.078.

Observations of the tumor in PEGylated PEDF treated animals revealed a surprising phenotype. SK-N-AS tumors in SCID mice typically recruit an abundant vasculature that infiltrates and surrounds the growing tumors, attaining the gross appearance of a peritumoral vascular ring. However, unlike tumors in untreated animals, which attained a domed appearance, tumors in the PEGylated PEDF treated animals were conspicuous for a visible reduction in vascularity at all time points and a flatter overall appearance. This is consistent with an anti-angiogenic effect.

Studies with the PEDF protein reported herein suggest that systemic delivery of PEDF protein reduces growth of a distal tumor. Prior histology data with intratumoral and intramuscular delivery of PEDF plasmid suggested that the effects of PEDF are at least in part due to inhibition of tumor-associated angiogenesis, as assessed by CD-31 immunostaining. Very low levels of PEDF protein (1 nanogram) yielded evidence of bioactivity when administered in the direct vicinity of the tumor. Studies of systemic administration suggest that locally bioactive levels may be reached with biweekly, i.v. delivery of 5 micrograms native protein, or 1 microgram PEGylated protein (approximately 200 micrograms/kg BW and 40 micrograms/kg, respectively). No evidence of morbidity or overt toxicity was noted in animals administered PEDF protein indicating that systemic administration of PEDF protein at these doses was generally well-tolerated.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method of improving an angiogenesis-inhibitory effect of an anti-angiogenic serpin, or anti-angiogenic fragment thereof, by covalently linking a polymer moiety to the serpin.

2. The method of claim 1 wherein the anti-angiogenic serpin is selected from the group consisting of: PEDF, maspin, antithrombin III, angiotensinogen, headpin, and combinations thereof.

3. The method of claim 2 wherein the anti-angiogenic serpin is PEDF.

4. The method of claim 2 wherein the anti-angiogenic serpin is maspin.

5. The method of claim 1, wherein the polymer comprises a polyethylene moiety.

6. The method of claim 5, wherein the polymer comprising a polyethylene moiety is selected from the group consisting of a polyethylene glycol and a poloxamer.

7. The method of claim 6, wherein the polymer is a polyethylene glycol.

8. The method of claim 7, wherein the polyethylene glycol is sulfonyl activated.

9. The method of claim 1, wherein the polymer has a molecular weight of about 20,000 or greater.

10. A composition comprising a modified anti-angiogenic serpin, or anti-angiogenic fragment thereof, wherein the serpin or fragment thereof is modified by covalent linkage to a polymer moiety.

11. The composition of claim 10, wherein the polymer is selected from the group consisting of a polyethylene glycol and a poloxamer.

12. The composition of claim 10, wherein the anti-angiogenic serpin is selected from the group consisting of: PEDF, maspin, antithrombin III, angiotensinogen, headpin, and combinations thereof.

13. The composition of claim 12, wherein the anti-angiogenic serpin is selected from the group consisting of PEDF, maspin, and combinations thereof.

14. The composition of claim 10, wherein the polymer has a molecular weight of about 20,000 or greater.

15. The composition of claim 11, wherein the polymer is a monomethoxy polyethylene glycol.

16. The composition of claim 15, wherein the monomethoxy polyethylene glycol is sulfonyl activated.

17. A method of inhibiting a disease having a pathological angiogenic component by administering in vivo an anti-angiogenic serpin, or fragment thereof, having a covalently linked polymer moiety.

18. The method of claim 17 wherein the anti-angiogenic serpin is selected from the group consisting of: PEDF, maspin, antithrombin III, angiotensinogen, headpin, and combinations thereof.

19. The method of claim 18 wherein the anti-angiogenic serpin is PEDF.

20. The method of claim 18 wherein the anti-angiogenic serpin is maspin.

21. The method of claim 17, wherein the disease is selected from the group consisting of diabetic retinopathy, age-related macular degeneration, rheumatoid arthritis, endometriosis, psoriasis, juvenile hemangioma, and cancer.

22. The method of claim 21 wherein the disease is cancer.

23. The method of claim 21, wherein the disease is age related macular degeneration or diabetic retinopathy.

24. The method of claim 17, wherein the polymer is selected from the group consisting of polyethylene glycol and poloxamers.

25. The method of claim 24, wherein the polymer is a polyethylene glycol.

26. The method of claim 25, wherein the polyethylene glycol is a sulfonyl activated polyethylene glycol.

27. The method of claim 24, wherein the polymer has a molecular weight of about 20,000 or greater.


SEQUENCE LISTING (2 sequences)

SEQ ID NO: 1 - protein, 418 aa, Homo sapiens [sequence omitted]

SEQ ID NO: 2 - protein, 375 aa, Homo sapiens
  Human Maspin (1)..(375): No canonical signal sequence
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP